(19) United States
(12) Patent Application Publication
(10) Pub. No.: US 2011/0177531 A1
(43) Pub. Date: Jul. 21, 2011

(54) Cell-Based Complement Activation Product Algorithm for Diagnosing Systemic Lupus Erythematosus

(75) Inventors: Thierry DERVIEUX, San Diego, CA (US); Michael GENDREAU, Poway, CA (US); Rong ZABLOCKI, San Diego, CA (US)

(73) Assignee: EXAGEN DIAGNOSTICS, INC., Albuquerque, NM (US)

(21) Appl. No.: 12/905,985

(22) Filed: Oct. 15, 2010

Related U.S. Application Data
(60) Provisional application No. 61/252,626, filed on Oct. 16, 2009.

(51) Int. Cl. G01N 33/53

(52) U.S. Cl. 435/7.92

(57) ABSTRACT

The present invention provides methods of diagnosing and monitoring systemic lupus erythematosus by measuring cell-based complement activation products (CB-CAPS) in a subject's blood. In particular, the invention describes a diagnostic method employing the measurement of multiple complement activation products on the surfaces of red blood cells, white blood cells, and platelets (e.g., EC4d, BC4d, PC4d, and ECR1). A diagnostic algorithm utilizing the determined levels of CB-CAPS is also disclosed.

### CROSS-REFERENCE TO RELATED APPLICATIONS

This application claims priority to U.S. Patent Application No. 61/252,626, filed on Oct. 16, 2009, the content of which is incorporated by reference herein in its entirety for all purposes.

### BACKGROUND OF THE INVENTION

Systemic Lupus Erythematosus (SLE) is an autoimmune disease, characterized by the production of unusual autoantibodies in the blood. These autoantibodies bind to their respective antigens, forming immune complexes which circulate and eventually deposit in tissues. This immune complex deposition causes chronic inflammation and tissue damage.

The precise reason for the abnormal autoimmunity that causes lupus is not known. Inherited genes, viruses, ultraviolet light, and drugs may all play some role. Genetic factors increase the tendency of developing autoimmune diseases, and autoimmune diseases such as lupus, rheumatoid arthritis, and immune thyroid disorders are more common among relatives of patients with lupus than the general population. Some scientists believe that the immune system in lupus is more easily stimulated by external factors like viruses or ultraviolet light. Sometimes, symptoms of lupus can be precipitated or aggravated by only a brief period of sun exposure.

Since patients with SLE can have a wide variety of symptoms and different combinations of organ involvement, no single test establishes the diagnosis of SLE. To help doctors improve the accuracy of diagnosis of SLE, eleven criteria were established by the American Rheumatism Association. These eleven criteria are closely related to the variety of symptoms observed in patients with SLE. When a person has four or more of these criteria, the diagnosis of SLE is strongly suggested. However, some patients suspected of having SLE may never develop enough criteria for a definite diagnosis. Other patients accumulate enough criteria only after months or years of observation. Nevertheless, the diagnosis of SLE may be made in some settings in patients with only a few of these classical criteria. Of these patients, a number may later develop other criteria, but many never do. The eleven criteria conventionally used for diagnosing SLE are:
1—malar over the cheeks of the face or “butterfly” rash
2—discoid skin rash: patchy redness that can cause scarring
3—photosensitivity: skin rash in reaction to sunlight exposure
4—mucus membrane ulcers: ulcers of the lining of the mouth, nose or throat
5—arthritis: two or more swollen, tender joints of the extremities
6—pleuritis/pericarditis: inflammation of the lining tissue around the heart or lungs, usually associated with chest pain with breathing
7—kidney abnormalities: abnormal amounts of urine protein or clumps of cellular elements called casts
8—brain irritation: manifested by seizures (convulsions) and/or psychosis
9—blood count abnormalities: low counts of white or red blood cells, or platelets
10—immunologic disorder: abnormal immune tests include anti-dsDNA or anti-Sm (Smith) antibodies, false positive blood tests for syphilis, anticardiolipin antibodies, lupus anticoagulant, or positive LE prep test, and
11—antinuclear antibody: positive ANA antibody testing

Although the criteria serve as useful reminders of those features that distinguish lupus from other related autoimmune diseases, they are unavoidably fallible. Determining the presence or absence of the criteria often requires interpretation. If liberal standards are applied for determining the presence or absence of a sign or symptom, one could easily diagnose a patient as having lupus when in fact they do not. Similarly, the range of clinical manifestations in SLE is much greater than that described by the eleven criteria and each manifestation can vary in the level of activity and severity from one patient to another. To further complicate a difficult diagnosis, symptoms of SLE continually evolve over the course of the disease. New symptoms in previously unaffected organs can develop over time. Because conventionally there is no definitive test for lupus, it is often misdiagnosed.

Monitoring disease activity is also problematic in caring for patients with lupus. Lupus progresses in a series of flares, or periods of acute illness, followed by remissions. The symptoms of a flare, which vary considerably between patients and even within the same patient, include malaise, fever, symmetric joint pain, and photosensitivity (development of rashes after brief sun exposure). Other symptoms of lupus include hair loss, ulcers of mucous membranes and inflammation of the lining of the heart and lungs which leads to chest pain.

Red blood cells, platelets and white blood cells can be targeted in lupus, resulting in anemia and bleeding problems. More seriously, immune complex deposition and chronic inflammation in the blood vessels can lead to kidney involvement and occasionally failure requiring dialysis or kidney transplantation. Since the blood vessel is a major target of the autoimmune response in lupus, premature strokes and heart disease are not uncommon. Over time, however, these flares can lead to irreversible organ damage. In order to minimize such damage, earlier and more accurate detection of disease flares would not only expedite appropriate treatment, but would reduce the frequency of unnecessary interventions. From an investigative standpoint, the ability to uniformly describe the “extent of inflammation” or activity of disease in individual organ systems or as a general measure is an invaluable research tool. Furthermore, a measure of disease activity can be used as a response variable in a therapeutic trial.

In an active disease state with severe inflammation, i.e., a flare, the expectation is that there will be a significant increase of anti-dsDNA and a significant decrease/consumption of serum C3 and C4 indicating serious activation of the complement system. If there is a positive response to therapy, it is expected that these levels will return to normal. It is widely accepted within the rheumatology community that the anti-dsDNA test is better for monitoring response to therapy than serum C3 and C4. The ds-DNA test is known to be highly specific for SLE, but suffers from a relatively low sensitivity because a significant percentage of confirmed SLE patients test negative for anti-dsDNA (approximately 40-50%).

In general, the complement system consists of a complex network of more than 30 functionally linked proteins that interact in a highly regulated manner to provide many of the effector functions of humoral immunity and inflammation, thereby serving as the major defense mechanism against bacterial and fungal infections. This system of proteins acts against invasion by foreign organisms via three distinct pathways: the classical pathway (in the presence of antibody), the alternative pathway (in the absence of antibody), and the lectin pathway. Once activated, the proteins within each pathway form a cascade involving sequential self-assembly into multimolecular complexes that perform various functions intended to eradicate the foreign antigens that initiated the response.

The classical pathway is usually triggered by an antibody bound to a foreign particle. It consists of several components that are specific to the classical pathway and designated C1, C4, C2, (in that order in the pathway). In the classical pathway, the first component C1q is bound to an antigen-antibody complex, activating the pathway. This event is followed by sequential activation of the two serine proteases C1r and C1s. Activated C1s has two substrates, the final two proteins of the classical pathway, namely C4 and C2. Protein C4 is cleaved into C4a and C4b. Protein C2 is cleaved to form C2a and C2b. Fragments C4b and C2a assemble to form C4b2a, which cleaves protein C3 into C3a and C3b, completing activation of the classical pathway.

Fragments C4b and C3b are subject to further degradation by Factor I. This factor cleaves C4b to generate C4d and also cleaves C3b, to generate iC3b followed by C3d. Thus, activation of the classical pathway of complement can lead to deposition of a number of fragments, including C4d and iC3b on immune complexes or other activating surfaces. These fragments are ligands for complement receptor type 1 (CR1) expressed on the surface of erythrocytes or red blood cells.

There have been inconsistent reports regarding complement proteins and SLE. One manifestation that has been reported in patients having SLE is a diminished expression of the complement receptor CR1 on erythrocytes (E-CR1) as compared to normal individuals (see, e.g., Ross et al. (1985), J. Immunol., Vol. 135: 2005; Corvetta et al. (1991), J. Rheumatol., Vol. 18: 1021). Iida et al. (J. Exp. Med. 155, 1427 (1982)) noted that the number of CR1 receptors on erythrocytes varied inversely with disease activity, with lower numbers occurring during periods of most severe manifestations of SLE, and higher numbers being observed during periods of remission in the same patients. Other studies seem to show there is no correlation.

The desired attributes of an effective and operational monitoring test or panel of tests for SLE include the ability to gauge disease activity, monitor and/or predict response to treatments, correlate with favorable outcomes and monitor and/or predict the onset of flares. Because no single test currently exists that exhibits all these attributes, there is a need in the art to develop additional sensitive and specific tests for diagnosing SLE and monitoring the therapeutic response in SLE patients.

### SUMMARY OF THE INVENTION

The present invention is based, in part, on the discovery that detection of a panel of cell-bound complement activation products (CB-CAPS) in blood samples can be used alone or in various combinations to provide a more sensitive and specific method of diagnosing and monitoring Systemic Lupus Erythematosus (SLE) in a subject. Accordingly, the present invention provides a method for diagnosing SLE in a subject by determining the level of at least one CB-CAP in a sample from the subject. In one embodiment, the method comprises determining the level of a first biomarker in a sample from a subject, wherein if the level of the first biomarker is within a first predetermined level, the subject is diagnosed with SLE. If the level of the first biomarker is outside the predetermined level, then the level of a second biomarker is determined, and if the level of the second biomarker is within a second predetermined level, the subject is diagnosed with SLE. In certain embodiments, the first biomarker is selected from the group consisting of BC4d, EC4d, and PC4d. In some embodiments, the second biomarker is a combination of an increased biomarker and a decreased biomarker, for instance, a ratio of the increased biomarker and the decreased biomarker. In one particular embodiment, the second biomarker is a ratio of EC4d and ECR1, BC4d and ECR1, or PC4d and ECR1.

In another embodiment, the method for diagnosing SLE comprises determining whether a sample from a subject is positive or negative for an antinuclear DNA antibody or anti-double stranded DNA test and determining the level of a first biomarker in a sample from a subject, wherein if the sample is positive for an antinuclear DNA antibody or anti-double stranded DNA test or if the level of the first biomarker is within a first predetermined level, the subject is diagnosed with SLE. If the sample is negative for an antinuclear DNA antibody or anti-double stranded DNA test or the level of the first biomarker is outside the predetermined level, then the level of a second biomarker is determined, and if the level of the second biomarker is within a second predetermined level, the subject is diagnosed with SLE. In some embodiments, the antinuclear DNA antibody or anti-double stranded DNA test is performed before, simultaneously with or after detection of the level of a first biomarker. In certain embodiments, the first biomarker is selected from the group consisting of BC4d, EC4d, and PC4d. In some embodiments, the second biomarker is a combination of an increased biomarker and a decreased biomarker, for instance, a ratio of the increased biomarker and the decreased biomarker. In one particular embodiment, the second biomarker is a ratio of EC4d and ECR1, BC4d and ECR1, or PC4d and ECR1.

In another embodiment of the invention, a method for diagnosing SLE comprises determining the level of a panel of biomarkers in a sample from a subject and determining whether the subject has SLE based on the level of the panel of biomarkers. In certain embodiments, the panel of biomarkers comprises (1) PC4d; (2) BC4d; (3) EC4d and PC4d; (4) EC4d and BC4d; (5) PC4d and BC4d; or (6) EC4d, PC4d, and BC4d. In one embodiment, the method further comprises determining the level of ECR1.

In some embodiments, the methods of the invention are performed in subjects that are negative for conventional tests, such as the antinuclear antibody test or anti-double stranded DNA test.

The present invention also includes a method for facilitating diagnosis of SLE. In one embodiment, the method comprises determining the level of a first biomarker in a sample from a subject, wherein if the level of the first biomarker is outside a predetermined level, then the level of a second biomarker is determined, and providing the level of the first biomarker and the second biomarker to an entity for diagnosis of SLE. In certain embodiments, the first biomarker is selected from the group consisting of BC4d, EC4d, and PC4d. In some embodiments, the second biomarker is a combination of an increased biomarker and a decreased biomarker, such as a ratio of the increased biomarker and the decreased biomarker. In one particular embodiment, the second biomarker is a ratio of EC4d and ECR1, BC4d and ECR1, or PC4d and ECR1.

In another embodiment, the method for facilitating diagnosis of SLE comprises determining whether a sample from a subject is positive or negative for an antinuclear DNA antibody or anti-double stranded DNA test and determining the level of a first biomarker in a sample from a subject, wherein if the antinuclear DNA antibody or anti-double stranded DNA test is negative or the level of the first biomarker is outside a predetermined level, then the level of a second biomarker is determined, and providing the level of the first biomarker and the second biomarker to an entity for diagnosis of SLE. In some embodiments, the antinuclear DNA antibody or anti-double stranded DNA test is performed before, simultaneously with or after detection of the level of a first biomarker. In certain embodiments, the first biomarker is selected from the group consisting of BC4d, EC4d, and PC4d. In some embodiments, the second biomarker is a combination of an increased biomarker and a decreased biomarker, such as a ratio of the increased biomarker and the decreased biomarker. In one particular embodiment, the second biomarker is a ratio of EC4d and ECR1, BC4d and ECR1, or PC4d and ECR1.

The present invention also provides kits and combinations of tests useful for diagnosing SLE according to the methods of the invention described herein. In one embodiment, the combination of tests comprises a first test for the level of EC4d, a second test for the level of PC4d, and a third test for the level of BC4d. In another embodiment, the combination further comprises at least one additional test for determining the level of ECR1. In still another embodiment, the combination further comprises at least one additional test for determining the presence of an antinuclear antibody or anti-double stranded DNA antibody in a sample. In yet other embodiments, the combination of tests comprises a first test for the presence or absence of antinuclear antibody or anti-double stranded DNA antibody and a second test for the level of one or more selected from the group consisting of EC4d, PC4d, or BC4d. In another embodiment, the combination further comprises at least one additional test for determining the level of ECR1.

A collection of results of the various levels of the biomarkers in a readable format for diagnosing SLE is also encompassed by the present invention. In one embodiment, the collection of results comprises the level of EC4d, the level of PC4d, and the level of BC4d. In another embodiment, the collection of results further comprises the level of ECR1. In yet another embodiment, the collection of results further comprises the presence or absence of antinuclear antibody or anti-double stranded DNA antibody. In another embodiment, the collection of results in readable format useful for diagnosing SLE comprises the presence or absence of antinuclear antibody or anti-double stranded DNA antibody and the level of the level of EC4d, PC4d, and/or the level of BC4d. In an additional embodiment, the collection of results further comprises the level of ECR1.

### BRIEF DESCRIPTION OF THE DRAWINGS

FIG. 1. Flow chart illustrating an algorithm for diagnosing SLE based on blood sample levels of cell-based complement activation products containing three detection tiers.

FIG. 2. Flow chart illustrating an algorithm for diagnosing SLE based on blood sample levels of cell-based complement activation products containing two detection tiers.

### DETAILED DESCRIPTION OF THE INVENTION

The present invention provides methods for the diagnosis and monitoring of disease activity and response to treatment in Systemic Lupus Erythematosus (SLE) using panels of biomarkers. In particular, the present invention includes the use of cell-bound complement activation products (CB-CAPS) as biomarkers to facilitate the diagnosis and monitoring of SLE in a subject. This panel of biomarkers can be used alone or in combination with traditional diagnostic assays (e.g., serum C3 levels, serum C4 levels, and anti-double-stranded DNA antibodies) to enhance the specificity and sensitivity of SLE diagnosis and disease monitoring.

Conventionally, a decrease in serum C3 and serum C4 levels has been used as a diagnostic marker for SLE because a decrease in these complement proteins was indicative of severe activation of the complement system. However, there are conflicting reports in the literature on the usefulness of these serum measurements of complement proteins to assess SLE status because serum C3 and C4 levels rarely quantitatively correlate with disease severity and there is a large variation of serum C3 and C4 levels in SLE patients that overlap with healthy subjects (Manzi et al. (2004) Lupus, Vol. 13:298-303). The presence of anti-dsDNA antibodies has been widely accepted as a highly specific test for diagnosing SLE and monitoring disease severity. However, there are a subset of confirmed SLE patients (approximately 40-50%) that test negative for anti-dsDNA antibodies. Thus, traditional measures for diagnosing and monitoring SLE in a subject lack accuracy and sensitivity, and improved methods of diagnosis using additional biomarkers are needed.

The present invention addresses this need by providing a panel of biomarkers that can be used alone or in combination with the traditional assays to facilitate the diagnosis and monitoring of SLE. As used herein, “biomarker” or “marker” refers to a biochemical molecule, macromolecule, or metabolite that can be measured in a biological sample from a subject that identifies SLE or status of inflammation (e.g., onset of a flare) in the subject. In particular, biomarkers can include products of the activation of the complement cascade deposited on the surface of cells found in the blood, such as red blood cells, white blood cells, and platelets (e.g., cell-based complement activation products or CB-CAPS). In certain embodiments, the panel of biomarkers includes EC4d (erythrocyte-bound C4d), ECR1 (erythrocyte complement receptor type 1), BC4d (B-lymphocyte-bound C4d), and PC4d (platelet-bound C4d).

“Diagnostic,” as used herein, means identifying the presence or nature of a pathologic condition, such as SLE. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In one embodiment, the present invention provides a method for diagnosing SLE comprising determining the level of a first biomarker in a sample from a subject. If the level of the first biomarker is within a predetermined level, then the subject is diagnosed with SLE. However, if the level of the first biomarker is outside the predetermined level, then the level of a second biomarker is determined in the sample. The subject is diagnosed with SLE if the level of the second biomarker is within a predetermined level. In some embodiments, the first biomarker is selected from the group consisting of BC4d, EC4d, and PC4d. In certain embodiments, the second biomarker is a combination of an increased biomarker and a decreased biomarker. For instance, in one particular embodiment, the second biomarker is a ratio of the increased biomarker and the decreased biomarker. Thus, in one embodiment, the second biomarker is a ratio of EC4d and ECR1. In another embodiment, the second biomarker is a ratio of BC4d and ECR1. In still another embodiment, the second biomarker is a ratio of PC4d and ECR1.

In other embodiments, the present invention provides a method for diagnosing SLE comprising determining whether a sample is positive or negative for an antinuclear antibody or anti-double stranded DNA test and determining the level of a first biomarker in a sample from a subject. If the antinuclear antibody or anti-double stranded DNA test is positive or level of the first biomarker is within a predetermined level, then the subject is diagnosed with SLE. However, if the antinuclear antibody or anti-double stranded DNA test is negative or the level of the first biomarker is outside the predetermined level, then the level of a second biomarker is determined in the sample. The subject is diagnosed with SLE if the level of the second biomarker is within a predetermined level. In some embodiments, the antinuclear DNA antibody or anti-double stranded DNA test is performed before, simultaneously with or after detection of the level of a first biomarker. In some embodiments, the first biomarker is selected from the group consisting of BC4d, EC4d, and PC4d. In certain embodiments, the second biomarker is a combination of an increased biomarker and a decreased biomarker. For instance, in one particular embodiment, the second biomarker is a ratio of the increased biomarker and the decreased biomarker. Thus, in one embodiment, the second biomarker is a ratio of EC4d and ECR1. In another embodiment, the second biomarker is a ratio of BC4d and ECR1. In still another embodiment, the second biomarker is a ratio of PC4d and ECR1.

In another embodiment, the present invention provides a method for diagnosing SLE comprising determining the level of a panel of biomarkers in a sample from a subject and determining whether the subject has SLE based on the level of the panel of biomarkers. In some embodiments, the panel of biomarkers comprises (1) PC4d; (2) BC4d; (3) EC4d and PC4d; (4) EC4d and BC4d; (5) PC4d and BC4d; or (6) EC4d, PC4d, and BC4d. In other embodiments, the panel of biomarkers is selected from the group consisting of (1) PC4d; (2) BC4d; (3) EC4d and PC4d; (4) EC4d and BC4d; (5) PC4d and BC4d; or (6) EC4d, PC4d, and BC4d. Thus, the diagnosis of SLE can be based on the measurement of PC4d level alone, BC4d level alone, a combination of EC4d and PC4d levels alone, a combination of EC4d and BC4d levels alone, a combination of PC4d and BC4d levels alone, or a combination of all three EC4d, BC4d, and PC4d levels. In certain embodiments, the method further comprises determining the level of ECR1. In such embodiments, ratios of one or more of the biomarkers in the panel to ECR1 levels can be calculated and compared to a pre-determined level to aid in diagnosis of SLE.

The present invention also provides a method for facilitating diagnosis of SLE. In one embodiment, the method comprises determining the level of a first biomarker in a sample from a subject, wherein if the level of the first biomarker is outside a predetermined level, then the level of a second biomarker is determined, and providing the level of the first and second biomarker to an entity for diagnosis of SLE. In another embodiment, the method comprises determining whether a sample is positive or negative for an antinuclear antibody or anti-double stranded DNA test and determining the level of a first biomarker in a sample from a subject, wherein if the sample is negative for the antinuclear antibody or anti-double stranded DNA test or the level of the first biomarker is outside a predetermined level, then the level of a second biomarker is determined, and providing the positive or negative antinuclear antibody or anti-double stranded DNA test result or the level of the first biomarker and the level of the second biomarker to an entity for diagnosis of SLE. The entity can be, but is not limited to, a clinical laboratory, a hospital, a clinician (e.g., a physician, a physician's assistant, a nurse practitioner), and an urgent care clinic.

In some embodiments, the first biomarker is selected from the group consisting of BC4d, EC4d, and PC4d. In other embodiments, the second biomarker is a combination of an increased biomarker and a decreased biomarker. In one embodiment, the second biomarker is a ratio of the increased biomarker and the decreased biomarker. For instance, in a particular embodiment the second biomarker is a ratio of EC4d and ECR1. In another embodiment, the second biomarker is a ratio of BC4d and ECR1. In still another embodiment, the second biomarker is a ratio of PC4d and ECR1.

Any of the diagnostic methods described herein can be used in combination with traditional diagnostic assays for SLE, such as serum C3 or C4 levels or presence of anti-dsDNA or antinuclear antibodies. As discussed previously, the diagnostic methods of the invention enhance the specificity and sensitivity of these conventional assays. The methods described herein are particularly useful when the conventional methods provide inconclusive results in diagnosing SLE. For instance, in one embodiment, the diagnostic methods of the invention are performed in a subject that is negative for the antinuclear antibody test or anti-double stranded DNA test.

The diagnostic methods described herein employ comparisons between a measured level of a biomarker and a pre-determined level or range. As used herein, a “pre-determined level” or “pre-determined range” refers to a value or range of values that can be determined from the quantity or amount (e.g., absolute value or concentration) of a particular biomarker measured in a population of control subjects (i.e. healthy subjects) or a population of subjects afflicted with an autoimmune disease other than SLE. A pre-determined level or pre-determined range can be selected by calculating the value or range of values that achieves the greatest statistical significance for a given set of amounts or quantities for a particular biomarker. In some embodiments, the pre-determined level can be based on the variance of a sample of biomarker quantities from a population of control/normal subjects. For instance, the pre-determined level can be at least 2, 3, 4, or 5 standard deviations above the normal range for a particular biomarker. In one embodiment, the pre-determined level is at least 6 standard deviations above the normal range for the biomarker. In some embodiments, a pre-determined level or pre-determined range can be a ratio of levels of two different biomarkers measured from all subjects (including Lupus patients). A pre-determined level or pre-determined range can also be determined by calculating a level or range of biomarker quantities for which greater than 50%, 60%, 70%, 75%, 80%, 85%, 90%, or 95% of patients having a quantity of biomarker within that level or range have SLE. Samples in which the level of biomarker does not fall within the pre-determined range or pre-determined level, may require the measurement of an additional biomarker before a diagnosis of SLE can be made.

Particularly suitable samples for use in the methods of the invention are blood samples. Blood samples are preferably treated with EDTA (ethylenediaminetetraacetate) to inhibit complement activation. Samples can be maintained at room temperature or stored at 4° C. In some embodiments, the whole blood sample may be fractionated into different components. For instance, in one embodiment, red blood cells are separated from other cell types in the sample by differential centrifugation. Analysis of complement activation products bound to erythrocytes (e.g., EC4d and ECR1) can be performed on the isolated red blood cells. In another embodiment, the platelet fraction is separated from other blood components to allow analysis of platelet-bound complement activation products, such as PC4d. Platelet isolation can be performed with methods known in the art, including differential centrifugation or immunoprecipitation using antibodies specific for platelets (e.g., CD42b).

In some embodiments, the white blood cells are isolated from other components of the blood sample. For example, white blood cells (the buffy coat) can be isolated from plasma and from red blood cells by centrifugation. Each type of white blood cell (e.g. lymphocyte, monocyte, etc.) can be isolated through the use of antibodies against known cell surface markers that are specific for that cell type. Antibodies against cell surface markers of white blood cells are known to those of skill in the art. For instance, monoclonal antibodies specific for cell surface markers CD3, CD4, CD8, and CD19 are commercially available and can be used to select lymphocytes. Analysis for complement activation products found on the surface of white blood cells, such as BC4d, can be performed in an isolated fraction of white blood cells.

The level (e.g., quantity or amount) of a particular biomarker can be measured in the sample using a variety of methods known to those of skill in the art. Such methods include, but are not limited to, flow cytometry, ELISA using red blood cell, platelet, or white blood cell lysates (e.g., lymphocyte lysates), and radioimmunoassay. In one embodiment, the determination of the level of C4d and CR1 is made using flow cytometric methods, with measurements taken by direct or indirect immunofluorescence using polyclonal or monoclonal antibodies specific for each of the molecules. Each of these molecules can be measured with a separate sample (e.g., red blood cell-, white blood cell-, or platelet-specific fractions) or using a single sample (e.g., whole blood).

The present invention also provides kits and combinations of tests for diagnosing SLE. In one embodiment, the present invention includes a combination of tests useful for diagnosing SLE comprising a first test for the level of EC4d, a second test for the level of PC4d, and a third test for the level of BC4d. In some embodiments, the combination further comprises at least one additional test for determining the level of ECR1. In other embodiments, the combination further comprises at least one additional test for determining the presence of antinuclear antibody or anti-double stranded DNA antibody in the sample. The kits or tests for determining the level of particular biomarkers include the various reagents for performing the measurements according to the methods described herein. For instance, in one embodiment, the kits or tests include reagents for performing immunofluorescence assays for each of the biomarkers, such as a conjugate of a monoclonal antibody specific for complement component C4d with a fluorescent moiety, and in some embodiments, a conjugate of a monoclonal antibody specific for complement receptor CR1 with a different fluorescent moiety. In certain embodiments, the kits or tests can include reagents for detecting antinuclear or anti-dsDNA antibodies, such as secondary antibodies labeled with a fluorescent tag, chemiluminescent tag, radiolabel tag or the like. Additionally, the kits can comprise such other material as may be needed in carrying out assays of this type, for example, buffers, radiolabelled antibodies, colorimeter reagents, instructions for separating different cell fractions from whole blood, and instructions for diagnosing SLE based on particular pre-determined levels of the biomarkers.

In another embodiment, the kits or tests include reagents for performing other standard assays for each of the biomarkers, such as ELISA or radioimmunoassays. In such embodiments, the kits or tests comprise monoclonal antibodies specific for C4d and CR1 conjugated with appropriate labels such as radioactive iodine, avidin, biotin or enzymes such as peroxidase. The kits can additionally comprise buffers, substrates for antibody-conjugated enzymes, instructions for separating different cell fractions from whole blood, and instructions for diagnosing SLE based on particular pre-determined levels of the biomarkers.

The methods of the invention as described herein can be carried out manually or may be used in conjunction with an automated system or computer. For instance, the methods can be performed using an automated system, in which a subject's blood sample is analyzed to make the necessary determination or determinations of levels of particular biomarkers, and the comparison with the pre-determined level or pre-determined range is carried out automatically by software appropriate for that purpose. Computer software, or computer-readable media for use in the methods of this invention include: a computer readable medium comprising: (a) code for receiving data corresponding to a determination of complement component C4d deposited on surfaces of red blood cells, platelets, or lymphocytes (e.g., B cells); (b) code for retrieving a pre-determined level for complement component C4d deposited on surfaces of such cells of individuals; and (c) code for comparing the data in (a) with the pre-determined level of (b) to make a determination whether an accurate SLE diagnosis can be made or whether additional measurements of other biomarkers are required. In some embodiments, the computer readable medium further comprises (d) code for receiving data corresponding to a determination of complement receptor CR1 deposited on surfaces of red blood cells; (e) code for retrieving a pre-determined level for complement receptor CR1 deposited on surfaces of red blood cells of individuals; and (f) code for comparing the data in (d) with the pre-determined levels of (e).

In certain embodiments of the invention, one or more pre-determined levels or pre-determined ranges of biomarker levels may be stored in a memory associated with a digital computer. After data corresponding to a determination of complement C4d, or complement receptor CR1 is obtained (e.g., from an appropriate analytical instrument), the digital computer can compare the measured biomarker data with one or more appropriate pre-determined levels or pre-determined ranges. After the comparisons take place, the digital computer can automatically calculate if the data is indicative of SLE diagnosis. Thus, the present invention also includes a collection of results in a readable format useful for diagnosing SLE comprising the level of EC4d, the level of PC4d, and the level of BC4d. In some embodiments, the collection of results further comprises the level of ECR1. In other embodiments, the collection of results further comprises the presence or absence of antinuclear antibody or anti-double stranded DNA antibody.

Accordingly, some embodiments of the invention may be embodied by computer code that is executed by a digital computer. The digital computer may be a micro, mini or large frame computer using any standard or specialized operating system such as a Windows based operating system. The code may be stored on any suitable computer readable media. Examples of computer readable media include magnetic, electronic, or optical disks, tapes, sticks, chips, etc. The code may also be written by those of ordinary skill in the art and in any suitable computer programming language including, C, C++, etc.

It is understood that the examples and embodiments described herein are for illustrative purposes only and that various modifications or changes in light thereof will be suggested to persons skilled in the art and are to be included within the spirit and purview of this application and scope of the appended claims. All references, publications, patents, and patent applications cited herein are hereby incorporated by reference in their entireties for all purposes.

### EXAMPLES

### Example 1

### A Cell-Based Complement Activation Products Algorithm for Diagnosing and Monitoring SLE

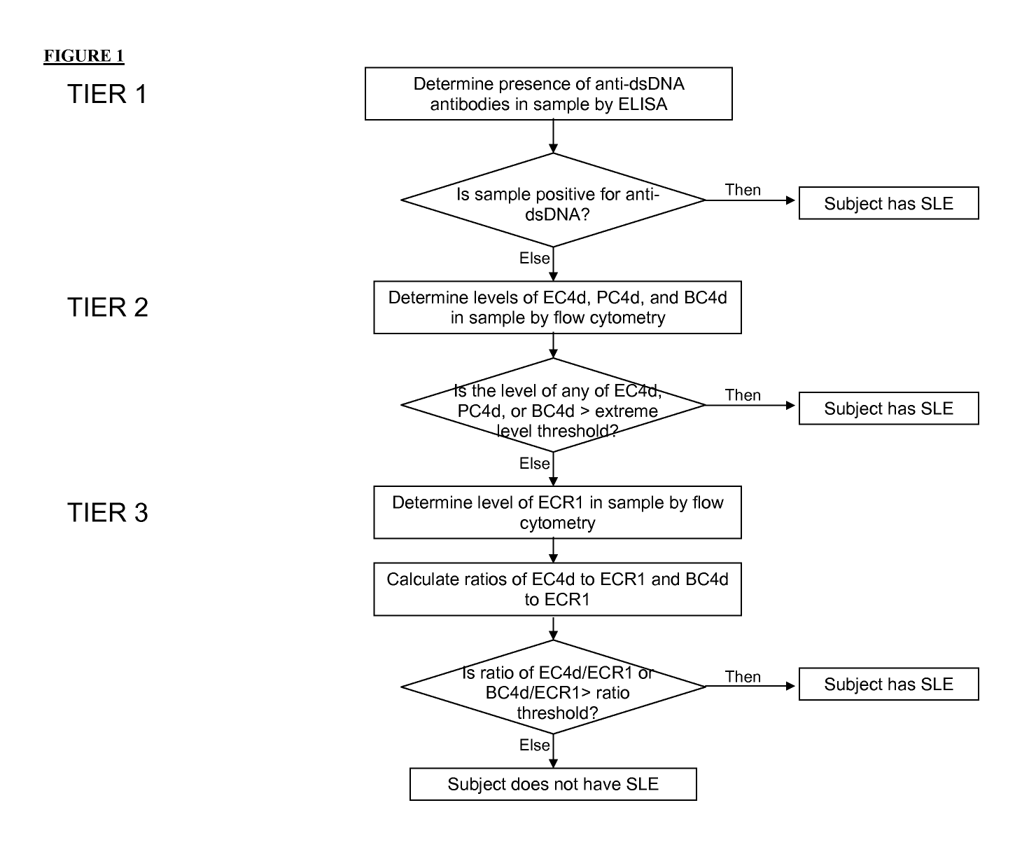

This Example outlines an algorithm to classify a patient as having a high probability of suffering from SLE (or not) based on two pieces of diagnostic information: 1) presence or absence of anti-double-strand DNA antibodies (DS-DNA) by a standard ELISA assay; and 2) flow cytometric determination of cell-bound complement activation product levels (e.g., CB-CAPS assay). A flow chart depicting the multi-step algorithm is shown in FIG. 1.

This multi-step approach involves three “tiers” of analysis. Tier 1 analysis involves DS-DNA analysis alone. Given the high specificity of double-strand DNA, a positive on DS-DNA is used to tentatively declare a patient positive for Lupus. CB-CAPS data for that patient is also collected for information related to monitoring and severity. A Tier 1 positive patient is displayed as DS-DNA+, suspect Lupus, with CB-CAPS analysis indicating whether the complement pattern is also consistent with Lupus. A patient Tier 1 positive has a result displayed with a specificity and diagnostic predictive accuracy value established for Tier 1 positives.

In the event that a patient is negative on DS-DNA, which represents approximately 40-50% of all confirmed SLE patients, then CB-CAPS analysis is used to evaluate the patient in Tier 2. An “extreme threshold” approach was used to develop a series of signal intensity cut-offs using three cell-based complement activation product (CB-CAP) markers, EC4D, PC4D, and BC4D, that are used to further characterize the probability of a patient being a Lupus patient. Tier 2 analysis determines if any of the individual levels of the three CB-CAP markers exceeds the “extreme threshold.” The extreme threshold was developed empirically from normal (e.g., healthy subjects) and/or subjects with an autoimmune disease other than SLE, and is designed to recognize a patient who has a complement-bound cellular signal that is in the range of 6-7 standard deviations above the normal range for that marker. Patients who have any of the three cellular markers at the extreme level are declared suspect Lupus patients at the specificity and diagnostic predictive accuracy established for Tier 2.

For the patient that does not exceed any of the extreme thresholds for the three markers, the algorithm then goes to a Tier 3 analysis. In Tier 3, a recursive partitioning approach was used to develop threshold ratios of the signal intensity for EC4D/ECR1 and BC4D/ECR1 to determine the probability that a patient has Lupus. EC4D and BC4D tend to be elevated in Lupus patients while ECR1 tends to be decreased. These ratios predict the likelihood of Lupus. Patients exceeding the established threshold for ratios are designated as suspect Lupus patients at the specificity and diagnostic predictive accuracy established for Tier 3.

By using this step-wise approach, we are able to assign a diagnostic accuracy value to the probability that a patient has Lupus or not. The confidence level of a positive result is related to the level at which the diagnosis is made. At the first tier, the very high specificity of DS-DNA gives a high confidence that this is in fact a Lupus patient. The algorithm can also use elevated levels of cell-bound complement to further enhance the confidence of this designation. At each Tier of analysis, a positive and negative predictive value is provided in the final report. The overall value of this algorithmic approach is to provide as much information as possible from diagnostic tests themselves, while maintaining as high a specificity as possible during the diagnostic analysis.

### Example 2

### A Cell-Based Complement Activation Products Algorithm for Diagnosing and Monitoring SLE

This Example outlines a second algorithm to classify a patient as having a high probability of suffering from SLE (or not), and provides a demonstration, based on two pieces of diagnostic information:

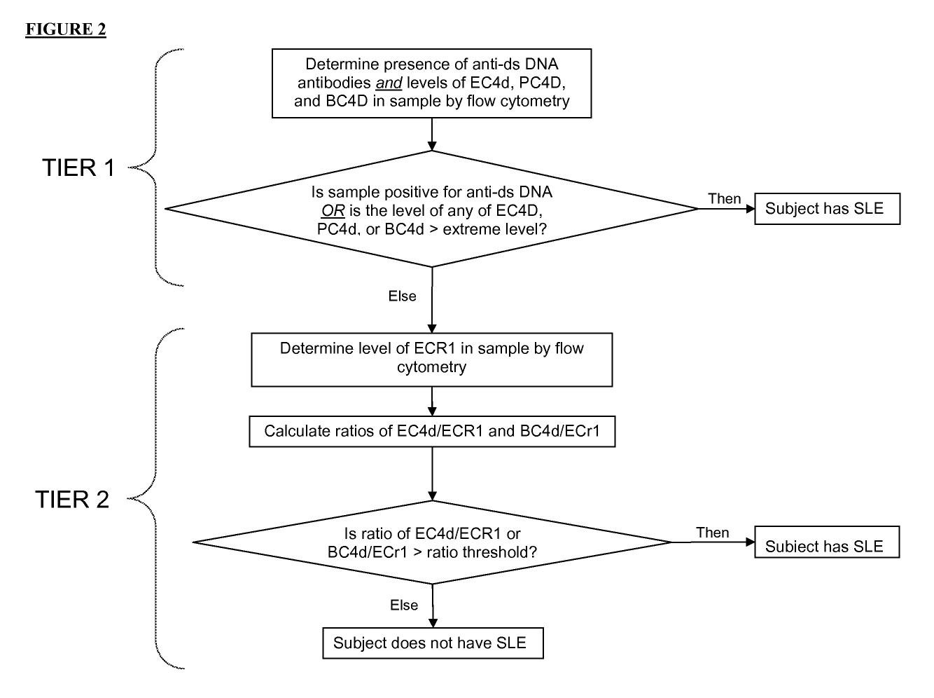

1) presence or absence of anti-double-strand DNA antibodies (DS-DNA) by a standard ELISA assay; and 2) flow cytometric determination of cell-bound complement activation product levels (e.g., CB-CAPS assay). A flow chart depicting the multi-step algorithm is shown in FIG. 2.

This multi-step approach involves two “tiers” of analysis. Tier 1 analysis involves both DS-DNA analysis and the signal intensity of three cell-based complement activation product (CB-CAP) markers, EC4D, PC4D, and BC4D. If the patient is positive on dsDNA testing or if the patient has an “extreme value” for the intensity of EC4D, PCD4, or BC4D (i.e. has a complement-bound cellular signal that is in the range of 6-7 standard deviations above the mean of that marker among non-lupus patients), then the patient is classified as positive for Lupus. A Tier 1 positive patient is displayed as either DS-DNA+, indicating a Lupus diagnosis, or with CB-CAPS results indicating a Lupus diagnosis, or both. A patient Tier 1 positive has a result displayed with a specificity and diagnostic predictive accuracy value established for Tier 1 positives.

Patients that are negative on Tier 1 testing (i.e. negative for DS-DNA, which represents approximately 40-50% of all confirmed SLE patients, and have no extreme value results in the CB-CAPS analysis) are then evaluated in Tier 2. In Tier 2, a recursive partitioning approach was used to develop threshold ratios of the signal intensity for EC4D/ECR1 and BC4D/ECR1 to determine the probability that a patient has Lupus. EC4D and BC4D tend to be elevated in Lupus patients while ECR1 tends to be decreased. These ratios predict the likelihood of Lupus. Patients with ratios outside the bounds of established threshold ratios are designated as Lupus patients at the sensitivity, specificity and diagnostic predictive accuracy established for Tier 1 and Tier 2 combined.

By using this step-wise approach, we are able to assign a diagnostic accuracy value to the probability that a patient has Lupus or not. The confidence level of a positive result is related to the tier at which the diagnosis is made. At the first tier, the very high specificity of DS-DNA gives a high confidence that a DS-DNA positive patient is in fact a Lupus patient. The elevated levels of any of the cell-bound complement further enhance the confidence of a Tier 1 positive patient designation as a Lupus patient. At both Tiers of analysis, a positive and negative predictive value is provided in the final report. The overall value of this algorithmic approach is to provide as much information as possible from the diagnostic tests themselves, while maintaining as high a specificity as possible during the diagnostic analysis.

We have applied the 2-Tiered algorithm to the group of patients summarized in Table 1 below:

TABLE 1
Study Subjects

| | | |
|---|---|---|
| 589 | 208 SLE | 145 dsDNA− |
| Total | | 63 ds DNA+ |
| Study | 381 All Others | 202 Normal Healthy |
| Subjects | | volunteers |
| | | 179 Other Rheumatic |
| | | Diseases |

The SLE patients were diagnosed according to the ACR Criteria for the Classification of SLE. The Other Rheumatic Diseases included Rheumatoid arthritis (67%), systemic sclerosis (12%), dermatomyosistis (5%), Sjogren's (5%), other vasculitis (4%), polymyositis (4%), and others (3%). All 589 study subjects were tested for dsDNA using a standard ELISA assay, and their levels of EC4D, BC4D, and PC4D were determined by flow cytometry, according to the Tier 1 strategy described above.

The results of Tier 1 are summarized in Table 2.

TABLE 2
Tier 1 Test Results

| Tier 1 | Disease - Negative | Disease - Positive |
|---|---|---|
| Test* | n (%) | n (%) |
| Test+ | 13 (3.41) | 103 (49.52) |
| Test− | 368 (96.59) | 105 (50.48) |
| Total | 381 | 208 |

*dsDNA Positive as defined by the manufacturer's instructions;
extreme value of EC4D in Other Rheumatic Disease + Normal Healthy group is 5.65 + 6 * 3.327 = 25.612;
extreme value for BC4D is 28.26 + 6 * 32.535 = 223.47;
extreme value for PC4D is 2.56 + 6 * 3.04 = 20.8

The Sensitivity for Tier 1 testing is 50%; the Specificity is 97%.

The Tier 1—negative study subjects were analyzed in Tier 2. The values for ECR1 were obtained by flow cytometry and the ratios of EC4D/ECR1 and BC4D/ECR1 were calculated. Recursive partitioning was used to establish the following rule for a positive test result:

### (EC4D/ECR1>0.59 and BC4D/ECR1>3.69) OR (EC4D/ECR1<=0.59 and BC4D/ECR1>4.48)=>Test (+)

The results of Tier 2 testing are summarized in Table 3.

TABLE 3
Tier 2 Test Results

| Tier 2 | Disease - Negative | Disease - Positive |
|---|---|---|
| Test* | n (%) | n (%) |
| Test+ | 17 (4.62) | 46 (43.81) |
| Test− | 351 (95.38) | 59 (56.19) |
| Total | 368 | 105 |

Sensitivity: 44%; Specificity: 95%

The combined sensitivity and specificity for Tier 1 and Tier 2 is:

### Specificity=351/381=92%

### Sensitivity=(103+46)/208=149/208=72%

The results of the Tier 1 and Tier 2 analysies are summarized in Table 4

TABLE 4
Summary of Two Tiered testing for Lupus diagnosis

| | | |
|---|---|---|
| Tier 1 | Sensitivity | 50% |
| (N = 589) | Specificity | 97% |
| Tier 2 | Sensitivity | 44% |
| (N = 473) | Specificity | 95% |
| Overall | Sensitivity | 72% |
| (N = 589) | Specificity | 92% |

It is understood that the disclosed invention is not limited to the particular methodology, protocols and materials described as these may vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A method for diagnosing Systemic Lupus Erythematosus (SLE) comprising
determining the level of a first biomarker in a sample from a subject,
wherein if the level of the first biomarker is within a first predetermined level, the subject is diagnosed with SLE,
wherein if the level of the first biomarker is outside the predetermined level, then the level of a second biomarker is determined, and
wherein if the level of the second biomarker is within a second predetermined level, the subject is diagnosed with SLE, and
wherein the first biomarker is selected from the group consisting of EC4d, PC4d, and BC4d and wherein the second biomarker is a combination of an increased biomarker and a decreased biomarker.

2. The method of claim 1, wherein the second biomarker is a ratio of the increased biomarker and the decreased biomarker.

3. The method of claim 1, wherein the second biomarker is a ratio of EC4d and ECR1, BC4d and ECR1, or PC4d and ECR1.

4. The method of claim 1, wherein the subject is negative for the antinuclear antibody test or anti-double stranded DNA test.

5. The method of claim 1, wherein the first predetermined level is at least 6 standard deviations above the normal range for the first biomarker.

6. A method for diagnosing Systemic Lupus Erythematosus (SLE) comprising determining the level of a panel of biomarkers in a sample from a subject and determining whether the subject has SLE based on the level of the panel of biomarkers, wherein the panel of biomarkers comprises 1) PC4d, 2) BC4d, 3) EC4d and PC4d, 4) EC4d and BC4d, 5) PC4d and BC4d, or 6) EC4d, PC4d, and BC4d.

7. The method of claim 6 further comprising determining the level of ECR1.

8. The method of claim 6, wherein the subject is negative for the antinuclear antibody test or anti-double stranded DNA test.

9. A method for facilitating diagnosis of SLE comprising
determining the level of a first biomarker in a sample from a subject,
wherein if the level of the first biomarker is outside a predetermined level, then the level of a second biomarker is determined, and
wherein the first biomarker is selected from the group consisting of EC4d, PC4d, and BC4d and wherein the second biomarker is a combination of an increased biomarker and a decreased biomarker, and
providing the level of the first biomarker and the second biomarker to an entity for diagnosis of SLE.

10. The method of claim 9, wherein the second biomarker is a ratio of the increased biomarker and the decreased biomarker.

11. The method of claim 9, wherein the second biomarker is a ratio of EC4d and ECR1, BC4d and ECR1, or PC4d and ECR1.

12. The method of claim 9, wherein the subject is negative for the antinuclear antibody test or anti-double stranded DNA test.

13. The method of claim 9, wherein the first predetermined level is at least 6 standard deviations above the normal range for the first biomarker.

14. A combination of tests useful for diagnosing SLE comprising 1) a first test for the level of EC4d, 2) a second test for the level of PC4d and 3) a third test for the level of BC4d.

15. The combination of claim 14, further comprising at least one additional test for the level of ECR1.

16. The combination of claim 14, further comprising at least one additional test for antinuclear antibody or anti-double stranded DNA antibody.

17. A collection of results in readable format useful for diagnosing SLE comprising 1) the level of EC4d, 2) the level of PC4d and 3) the level of BC4d.

18. The collection of claim 17, further comprising the level of ECR1.

19. The collection of claim 17, further comprising the presence or absence of antinuclear antibody or anti-double stranded DNA antibody.

20. A method for diagnosing Systemic Lupus Erythematosus (SLE) comprising
determining whether a sample from a subject is positive or negative for an antinuclear antibody test or anti-double stranded DNA test and
determining the level of a first biomarker in a sample from a subject,
wherein if the sample is positive for an antinuclear antibody test or anti-double stranded DNA test or the level of the first biomarker is within a first predetermined level, the subject is diagnosed with SLE, and
wherein if the sample is negative for an antinuclear antibody test or anti-double stranded DNA test or the level of the first biomarker is outside the predetermined level, then the level of a second biomarker is determined, and
wherein if the level of the second biomarker is within a second predetermined level, the subject is diagnosed with SLE, and
wherein the first biomarker is selected from the group consisting of EC4d, PC4d, and BC4d and wherein the second biomarker is a combination of an increased biomarker and a decreased biomarker.

21. The method of claim 20, wherein the second biomarker is a ratio of the increased biomarker and the decreased biomarker.

22. The method of claim 20, wherein the second biomarker is a ratio of EC4d and ECR1, BC4d and ECR1, or PC4d and ECR1.

23. The method of claim 20, wherein the first predetermined level is at least 6 standard deviations above the normal range for the first biomarker.

24. A method for facilitating diagnosis of SLE comprising
determining whether a sample from a subject is positive or negative for an antinuclear antibody test or anti-double stranded DNA test and
determining the level of a first biomarker in a sample from a subject, wherein if the sample is negative for an antinuclear antibody test or anti-double stranded DNA test or if the level of the first biomarker is outside a predetermined level, then the level of a second biomarker is determined, and
wherein the first biomarker is selected from the group consisting of EC4D, PC4d and BC4d and wherein the second biomarker is a combination of an increased biomarker and a decreased biomarker, and
providing the positive or negative antinuclear antibody test or anti-double stranded DNA test result or level of the first biomarker results and the level of the second biomarker to an entity for diagnosis of SLE.

25. The method of claim 24, wherein the second biomarker is a ratio of EC4d and ECR1, BC4d and ECR1, or PC4d and ECR1.

26. The method of claim 24, wherein the first predetermined level is at least 6 standard deviations above the normal range for the first biomarker.

27. A combination of tests useful for diagnosing SLE comprising 1) a first test for the presence or absence of antinuclear antibody or anti-double stranded DNA antibody and 2) a second test for the level of one or more biomarkers selected from the group consisting of EC4d, PC4d, and BC4d.

28. The combination of claim 27 further comprising at least one additional test for the level of ECR1.

29. A collection of results in readable format useful for diagnosing SLE comprising 1) the presence or absence of antinuclear antibody or anti-double stranded DNA antibody and 2) the level of one or more biomarkers selected from the group consisting of EC4d, PC4d, and BC4d.

30. A collection of claim 29, further comprising the level of ECR1.

31. A collection of results in readable format useful for diagnosing SLE comprising 1) the level of one or more selected from the group consisting of EC4d, PC4d, and BC4d and 2) the level of ECR1.

32. The collection of claim 31, further comprising the presence or absence of antinuclear antibody or anti-double stranded DNA antibody.
